(19) United States
(12) Patent Application Publication
(10) Pub. No.: US 2022/0176028 A1
(43) Pub. Date: Jun. 9, 2022

(54) BLOOD PERFUSION DEVICE

(71) Applicant: Jörg GERLACH, Berlin (DE)

(72) Inventor: Reinhard BORNEMANN, Bielefeld (DE)

(21) Appl. No.: 17/602,226

(22) PCT Filed: Apr. 3, 2020
(86) PCT No.: PCT/EP2020/059525

§ 371 Date: Oct. 7, 2021

(30) Foreign Application Priority Data
Apr. 11, 2019 (EP) PCT/EP2019/059331

(51) Int. Cl. A61M 1/34; A61M 60/279; A61M 60/113; A61M 60/36; A61M 1/36

(52) U.S. Cl. CPC A61M 1/3496; A61M 60/279; A61M 60/113; A61M 2202/0415; A61M 1/367; A61M 1/3427; A61M 1/3455; A61M 60/36

(57) ABSTRACT

The present invention relates to various methods of treatment using a novel blood perfusion device. The blood perfusion device comprises a perfusion chamber comprising at least one compartment A and at least one compartment B, compartment A comprising a first opening which is in direct fluid communication to a second opening, wherein the first opening of compartment A is in direct fluid communication to a first port of the perfusion chamber and the second opening of compartment A is in direct fluid communication to a second port of the perfusion chamber; and compartment B comprising a first opening which is in direct fluid communication to a second opening, wherein the first opening of compartment B is in direct fluid communication to a third port of the perfusion chamber and the second opening of compartment B is in direct fluid communication to a fourth port of the perfusion chamber, wherein compartment A is separated from compartment B by at least one membrane, said membrane being configured to prevent cells from crossing the membrane.

### TECHNICAL FIELD

The present invention relates to various methods of treatment using a novel blood perfusion device. The blood perfusion device comprises a perfusion chamber comprising at least one compartment A and at least one compartment B, compartment A comprising a first opening which is in direct fluid communication to a second opening, wherein the first opening of compartment A is in direct fluid communication to a first port of the perfusion chamber and the second opening of compartment A is in direct fluid communication to a second port of the perfusion chamber; and compartment B comprising a first opening which is in direct fluid communication to a second opening, wherein the first opening of compartment B is in direct fluid communication to a third port of the perfusion chamber and the second opening of compartment B is in direct fluid communication to a fourth port of the perfusion chamber, wherein compartment A is separated from compartment B by at least one membrane, said membrane being configured to prevent cells from crossing the membrane.

### BACKGROUND

During gestation, a mother shares any metabolic support with the growing child through the mother's placenta. The placenta provides a functional interface between the blood circuit of the mother and the blood circuit of the fetus. It represents a blood cell barrier (including for immune competent cells) and a mass exchanger between maternal and child blood plasma, for all molecules smaller than blood cells. This mass exchanger, the placenta, is connected via an artery and a vein on the mother's side and an artery and a vein on the child's side, the vascular walls of two blood capillary systems in the placenta form a membrane in between that is not permeable for blood cells, only for plasma. This interface allows the unborn child to fully benefit from the mothers liver, kidney, lung and other organ functions. When a child birth occurs prematurely, that life-line is irreversibly interrupted, the organ support function provided by the mother is no longer available for the child. Since the newborn's organs are not yet fully developed, multiple severe and life threatening situations are often the inevitable result.

The clinical therapy of acute hepatic failure in children is only one example that remains a challenge. Acute liver failure is expressed with severe encephalopathy, coagulopathy, and subsequent multisystem organ failure, resulting in a high death rate. When a transplant donor organ, or partial organ, is available, liver transplantation is considered the best option, with long-term 1-year survival rates exceeding 88% (Singer, Andrew L. et al. “Role of Plasmapheresis in the Management of Acute Hepatic Failure in Children.” Annals of Surgery 234.3 (2001): 418-424)). But liver transplantation or partial liver transplantation in prematurely born children is not feasible.

The above problems, in this particular situation, could be avoided by replacing the mother's placenta with an extracorporeal “artificial placenta” that allows the child post premature birth to continue benefiting, temporarily, from the extracorporeal organ function of the mother.

Accordingly, the present invention provides a medical device for temporary extracorporeal blood perfusion, with a perfusion chamber that functions as a mass exchanger between two blood circuits connected to the device.

### SUMMARY

The present invention relates to a blood perfusion device comprising a perfusion chamber (1) comprising at least one compartment A and at least one compartment B, (a) compartment A (4) comprising a first opening which is in direct fluid communication to a second opening, wherein the first opening of compartment A is in direct fluid communication to a first port (2) of the perfusion chamber and the second opening of compartment A is in direct fluid communication to a second port (3) of the perfusion chamber; and (b) compartment B (7) comprising a first opening which is in direct fluid communication to a second opening, wherein the first opening of compartment B is in direct fluid communication to a third port (5) of the perfusion chamber and the second opening of compartment B is in direct fluid communication to a fourth port (6) of the perfusion chamber; wherein compartment A is separated from compartment B by at least one membrane, said membrane being configured to prevent cells from crossing the membrane. Preferably, said membrane is a semipermeable membrane.

In embodiment, said ports can be connected outside of the perfusion chamber to tubes (9) and/or to a pump (14).

In another embodiment, the membrane of the blood perfusion device of the present invention is a hollow fiber membrane system, wherein compartment A is inside a first hollow fiber membrane system that connects the first port (2) and the second port (3) and compartment B is inside a second hollow fiber membrane system that connects the third port (5) and the fourth port (6). In one embodiment, the first hollow fiber membrane system comprising compartment A and the second hollow fiber membrane system comprising compartment B are within the same compartment, i.e. compartment C (15). The perfusion chamber may comprise an additional fifth port (10) and an additional sixth port (11), wherein the fifth port is in direct fluid communication to a first opening of compartment C and the sixth port is in direct fluid communication to a second opening of compartment C, wherein said ports are connected outside the perfusion chamber to a circulation system (12) comprising a pump (13) for adding recirculation flow through the perfusion chamber

In another embodiment, the blood perfusion device of the present invention comprises a compartment A and a compartment B which are separated by at least one planar membrane. Said compartments A and B can be separated, for example, by two planar membranes, thus forming compartment C (15) between said planar membranes. The perfusion chamber may comprise an additional fifth port (10) and an additional sixth port (11) wherein the fifth port is in direct fluid communication to a first opening of compartment C and the sixth port is in direct fluid communication to a second opening of compartment C, wherein said ports are connected outside the perfusion chamber to a circulation system (12) comprising a pump (13) for adding recirculation flow through the perfusion chamber

In one aspect, the blood perfusion device of the present invention is equipped with a detector. In one embodiment, the perfusion chamber (1), the circulation system (12) or the pump (13) comprises one or more detectors. The detector can be, for example, a detector for detecting pressure, rate of flow, temperature, oxygen, carbon dioxide, pH, CRP red color, hemoglobin, or blood cells.

In one embodiment, the membrane of the blood perfusion device of the present invention is semipermeable and preferably has a molecular weight-cutoff of 10 kDa-800 kDa, more preferably of up to 400 kDa.

In another embodiment, the membrane comprises a material selected from the group consisting of polysulfone, polyethersulfone, polypropylene, polyamide, and cellulose, preferably polyethersulfone. The membrane is preferably microporous.

In another embodiment, the casing of the perfusion chamber comprises a material selected from the group consisting of pvc, polypropylene, polyurethane, polyamide, polyethylene, polyethersulphone, polystyrole, and silicone rubber.

In one embodiment, the first hollow fiber membrane system comprising compartment A and the second hollow fiber membrane system comprising compartment B, and optionally the axis connecting the fifth port (10) and the sixth port (11) are in parallel orientation.

In another embodiment, the perfusion chamber of the present invention comprises one or more additional ports for direct injection of medical drugs, additional plasma or plasma proteins, and sensors that measure physical properties, chemical properties and substances in the device, wherein said additional ports are preferably in direct fluid communication to compartment C.

In another embodiment, the blood perfusion device of the present invention comprises a control unit. The control unit can be configured to provide a flow rate of 1-350 ml/min at the first port (2) and the second port (3), and at the third port (5) and the fourth port (6). Preferably, the flow rate is adjusted by one or more pumps (14).

In one embodiment, the control unit of the blood perfusion device of the present invention is configured to provide a difference between the flow rate through compartment A and compartment B of less than 1% to avoid net plasma volume transfer from compartment A to the compartment B.

In one embodiment, the control unit of the blood perfusion device of the present invention is configured to provide a difference between the flow rate through compartment A and compartment B which is between 1%-20% for generating net plasma volume transfer from compartment A to compartment B.

In one embodiment, the blood perfusion device of the present invention comprises one or more ports which are made from or comprise pvc, polypropylene, polyamide, polyethylene, or polyethersulphone.

In one embodiment, the blood perfusion device of the present invention comprise a pump (13) or a pump (14), said pump being selected from the group consisting of centrifugal pump, fingerprint tubing pump, and roller tubing pump.

In one embodiment, the blood perfusion device or the control unit of the present invention is configured to allow a flow through compartment A which is counter-directional to the flow through compartment B.

In another embodiment, the blood perfusion device or the control unit is configured so that the first opening of compartment A which is in direct fluid communication to the first port (2) is an inlet of compartment A and the second opening of compartment A which is in direct fluid communication to the second port (3) is an outlet of compartment A, and the first opening of compartment B which is in direct fluid communication to the third port (5) is an inlet of compartment B and the second opening of compartment B which is in direct fluid communication to the fourth port (6) is an outlet of compartment B.

In another embodiment, the blood perfusion device or the control unit is configured so that the first opening of compartment A which is in direct fluid communication to the first port (2) is an inlet of compartment A and the second opening of compartment A which is in direct fluid communication to the second port (3) is an outlet of compartment A, and the first opening of compartment B which is in direct fluid communication to the third port (5) is an outlet of compartment B and the second opening of compartment B which is in direct fluid communication to the fourth port (6) is an inlet of compartment B.

In another embodiment, the perfusion chamber of the blood perfusion device of the present invention comprises a hollow fiber membrane system, wherein compartment A is inside a first hollow fiber membrane system that connects the first port (2) and the second port (3) and compartment B is inside a second hollow fiber membrane system that connects the third port (5) and the fourth port (6). In one embodiment, the first hollow fiber membrane system comprising compartment A and the second hollow fiber membrane system comprising compartment B are within the same compartment, i.e. compartment C (15). The perfusion chamber may comprise an additional fifth port (10) and an additional sixth port (11), wherein the fifth port is in direct fluid communication to a first opening of compartment C and the sixth port is in direct fluid communication to a second opening of compartment C, wherein said ports are connected outside the perfusion chamber to a circulation system (12) comprising a pump (13) for adding recirculation flow through the perfusion chamber, preferably through compartment C of the perfusion chamber.

In one embodiment, the blood perfusion device or the control unit of the blood perfusion device is configured so that the first opening of compartment C which is in direct fluid communication to the fifth port (10) is an inlet of compartment C and the second opening of compartment C which is in direct fluid communication to the sixth port (11) is an outlet of compartment C.

In one embodiment, the blood perfusion device or the control unit of the blood perfusion device is configured so that the first opening of compartment C which is in direct fluid communication to the fifth port (10) is an outlet of compartment C and the second opening of compartment C which is in direct fluid communication to the sixth port (11) is an inlet of compartment C.

In one embodiment, the blood perfusion device of the present invention or the control is configured so that the flow rate at the first port (2) and the second port (3), and/or at the third port (5) and the fourth port (6) is 1-350 ml/min. In another embodiment, the flow rate for a subject which is a baby is 1-50 ml/min, the flow rate for a subject which is a child is 10-150 ml/min, the flow rate for a subject which is an adult is 20-350 ml/min, preferably 100-150 ml/min.

In one embodiment, the flow rate is adjusted by a pump (14).

In another embodiment, the flow rate through the circulation system (12) is preferably adjusted by the pump (13).

The present invention also relates to a method of operating the blood perfusion device of the present invention. In one embodiment, the method comprises connecting a first and a second subject to the blood perfusion device. The method comprises operating the blood perfusion device in a manner allowing the blood of the first subject to enter into compartment A (4) of the perfusion chamber and allowing the blood of the second subject to enter into compartment B (7) of the perfusion chamber. The method can be used in a method of treating or preventing a condition, preferably a condition described herein below.

The present invention also relates to a method of treatment or prevention of a condition, said method comprising (a) connecting a first subject to the first port (2) and the second port (3) of the blood perfusion device of the present invention and connecting a second subject to the third port (5) and the fourth port (6) of said blood perfusion device; and (b) allowing the blood of the first subject to enter into compartment A (4) of the perfusion chamber and allowing the blood of the second subject to enter into compartment B (7) of the perfusion chamber, wherein said treatment comprises mass exchange between blood plasma of the first subject and the second subject, wherein the first subject is preferably a healthy subject and the second subject is in need of treatment.

In one embodiment, the second subject is in need of blood plasma treatment.

In another embodiment, the first subject is characterized by normal organ functions and normal plasma composition and the second subject is characterized by at least one deficient organ function and a deficient blood plasma composition.

In one embodiment, the deficient organ of the second subject is the kidney, the liver or the lung. Preferably, the deficient organ of the second subject is the a failing kidney in acute or chronic renal failure, the liver in acute, chronic or acute-on chronic hepatic failure or the lung with impaired oxygenation function of carbon dioxide removal function.

In one embodiment, the liver is affected from an acute, acute-on chronic or chronic liver disease associated with one or more aberrant liver-related blood parameter, wherein the parameter is preferably selected from the group consisting of ammonia, bilirubin and pH.

In one embodiment, the kidney of the second subject is affected from an acute or chronic kidney disease associated with one or more aberrant kidney-related blood parameter, wherein the parameter is preferably selected from the group consisting of urea, water creatine and electrolytes, including potassium, sodium, chloride, magnesium, calcium.

In one embodiment, the second subject is in need of renal dialysis or hemodialysis.

In one embodiment, the lung of the second subject is affected from an acute, acute on or chronic lung disease associated with one or more aberrant lung-related blood parameter, wherein the parameter is preferably selected from the group consisting pH, oxygen and carbon dioxide.

In one embodiment, the second subject is affected from a multi-organ failure.

In one embodiment, the blood plasma of the second subject is characterized by an aberrant level of a hormone, mediator and/or cytokine or an aberrant level of cytokine regulating organ function, as known from sepsis, shock and multi-organ failure, including non-septic multi-organ failure after trauma.

In one embodiment, the condition which is treated or prevented is or includes a condition selected from the group consisting of a weakness of bones with bone loss and bone fractures (osteoporosis), a loss of muscle strength or muscle tissue (muscular dystrophies), a loss of connective tissue strength (joint cartilage weakness), a loss of hair strength and thickness, a loss of skin strength and thickness, an ageing-related condition (such as weakness of the muscular skeletal system), an integrity weakness of tissues and organs (such as cartilage, joints, tendons), an under-function of tissues and organs (such as kidney, lung, liver), a mal-function of tissues and organs (such as liver and the white blood cell related mediator homeostasis), a non-function of tissues and organs (such as kidney, lung, liver), a deregulation of oncotic pressure (e.g. as a consequence of liver failure), a deregulation of osmolarity (e.g. as a consequence of kidney failure, an aberrant level of pH (e.g. as a consequence of liver failure) and an aberrant level of electrolytes (e.g. as a consequence of kidney failure).

In one embodiment, the condition is or includes a condition involving in the second subject an aberrant level of a mediator such as a cytokine, wherein the condition is preferably shock, septic shock, and multi-organ failure.

In another embodiment, the second subject is in a medical situation approaching one or several of the above conditions and the treatment is performed to prevent one or several of the above conditions, such as multi-organ failure.

In another embodiment, the second subject is a preterm baby.

In another embodiment, the second subject is a victim in a mass trauma.

In another embodiment, the second object is a victim in military combat situations.

### BRIEF DESCRIPTION OF THE DRAWINGS

FIG. 1A: The figure shows the simplest version of a blood perfusion chamber according to the present invention. A chamber divided by a membrane into two separate compartments (4) and (7) can be connected with blood tubing and blood can be pumped through each one of the compartments. In this case two independent blood circuits are connected: the first circuit starting at port (2) and ending at port (3) and the second starting at port (5) and ending at port (6). The large arrows indicate the blood flow direction (in this illustration with co-current flow operation). The chamber is separated by a semipermeable membrane, in this case a flat sheet membrane, in the middle vertically of the drawing of FIG. 1A, and the membrane separates the blood flow on both sides of the chamber and thus in both blood circuits, left and right. This membrane features a molecular eight cut-off of for example around MW400000, and therefore prevents a passing of much larger blood cells, including blood-group carrying erythrocytes and immune cells, from one side to the other but allows a passing of the blood plasma between each parts of the chamber. Therefore, an immune-barrier is provided between both blood circuits, while both blood circuits can freely exchange the molecules in the plasma—that are smaller than blood cells and also smaller than for example MW 400000. An important molecule to be exchanged is, e.g., the carrier protein albumin that has a MW of 60000 and that, thus, can freely pass the membrane from both sides of the blood circuit back and forth.

FIG. 1B: The figure shows an enhancement of the configuration shown in FIG. 1A. The chamber comprises two membranes which serve the same purpose and create a new compartment (15). However, two advantages can be described: a) a blood cell leakage in a first membrane does not lead to a blood cell loss into the second blood circuit and furthermore the blood cell loss can be detected immediately in this compartment (15), e.g. by detecting the red blood cell color with a color indicator in the compartment; b) by adding a third circuit (14), starting at port (10) and ending at port (11), an enhanced flow of the blood plasma in the new compartment (15) between the membranes can be enabled and thus the plasma mass exchange between the two blood circuits is enhanced. A blood cell sensor can be connected to or inserted into the third circuit (14) or to compartment (15).

FIG. 2A-2G: This figure shows a blood perfusion chamber comprising two hollow fiber membrane systems instead of the flat sheet membranes shown in FIG. 1A-1B. Various exemplary configurations are shown in 2A to 2G.

Abbreviations: P=pump, S=sensor. “(4)” corresponds to “compartment A” of the present teaching, “(7)” corresponds to “compartment B” of the present teaching and “(15)” corresponds to “compartment C” of the present teaching.

### DETAILED DESCRIPTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described. These elements are listed with specific embodiments. However, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “about” means approximately or nearly, and in the context of a numerical value or range set forth herein in one embodiment means ±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

In the following, definitions will be provided which apply to all aspects of the present disclosure. The following terms have the following meanings unless otherwise indicated. Any undefined terms have their art recognized meanings.

As used herein, the term “blood perfusion device” refers to a medical device, apparatus, or instrument that is designed to support blood plasma exchange between two subjects which are connected to the device.

The blood perfusion device of the present teaching comprises at least one perfusion chamber. As used herein, the terms “one or more” and “at least one” generally mean at least 1, at least 2, at least 3, at least 4, at least 5, at least 10, at least 15 or at least 50. In some cases, however, a limitation to up to 10, 20, 30, 40 or 50 may be desirable.

The term “perfusion chamber” refers to a component of the blood perfusion device of the present teaching which facilitates blood plasma exchange between two subjects. Typically, the blood perfusion chamber is inside a housing and comprises at least two or three compartments. In other words, the space inside the perfusion chamber is subdivided or split into separate “rooms” or “compartments”, wherein a compartment is typically separated from a neighboring compartment by at least one membrane. For example, the perfusion chamber may comprise three compartments. A first compartment can be considered as compartment of type A (“compartment A”), a second compartment can be considered as compartment of type B (“compartment B”), a third compartment can be considered as compartment of type C (“compartment C”), etc. Preferably, the membrane is permeable for smaller subcellular structures such as proteins but non-permeable for larger structures such as cells. In operative state of the blood perfusion device, the compartments of the perfusion chamber are filled with a fluid, typically with a physiological buffer or with blood or blood plasma.

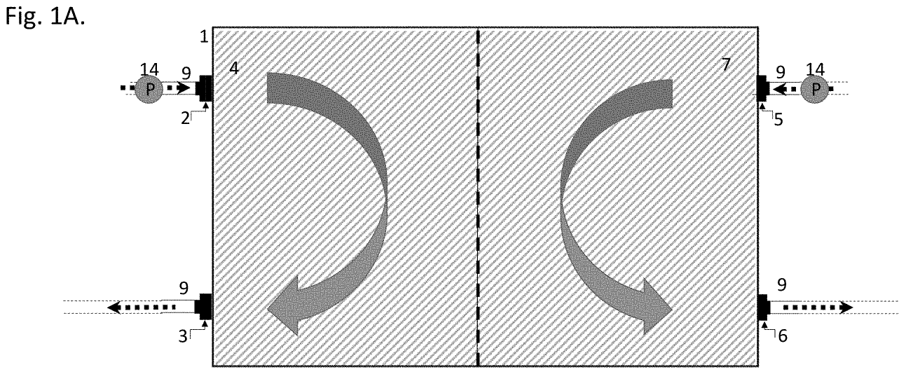

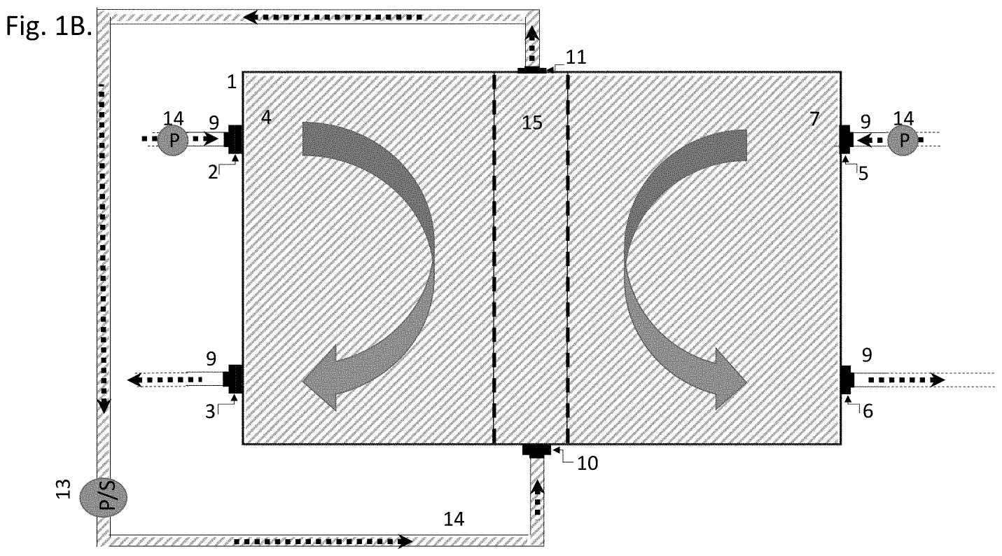

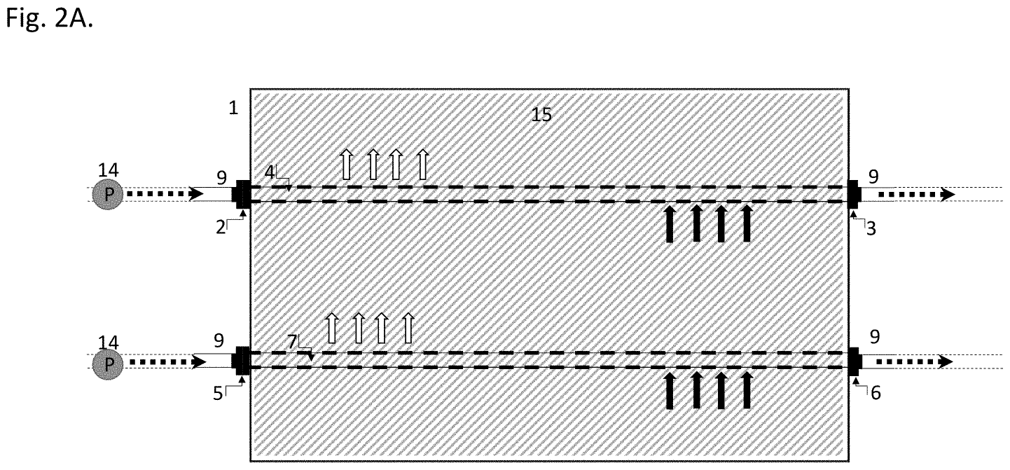

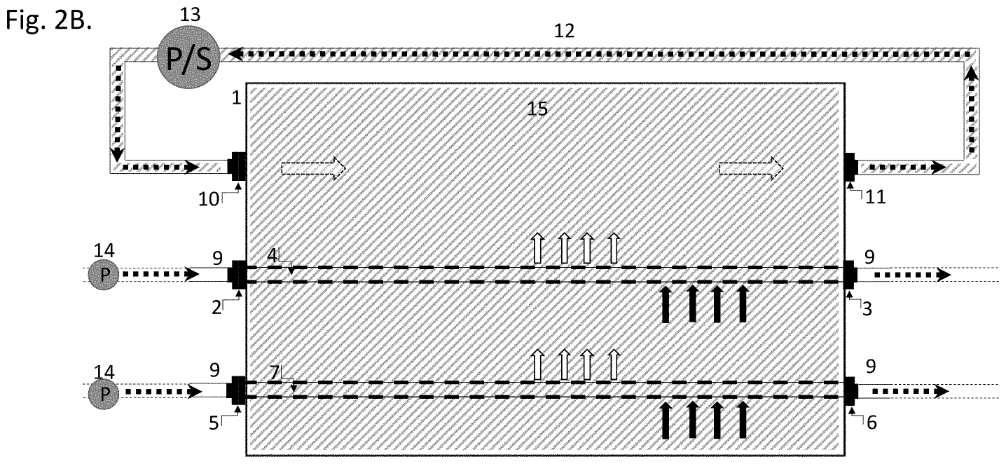

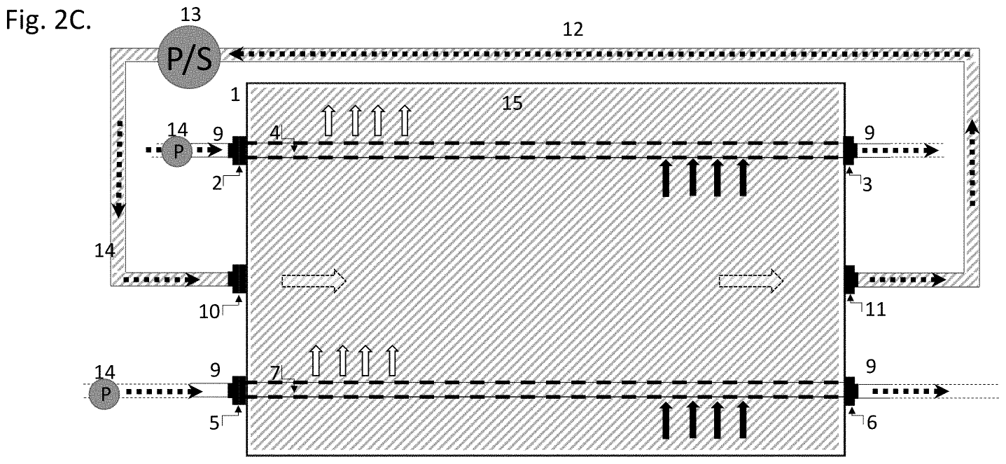

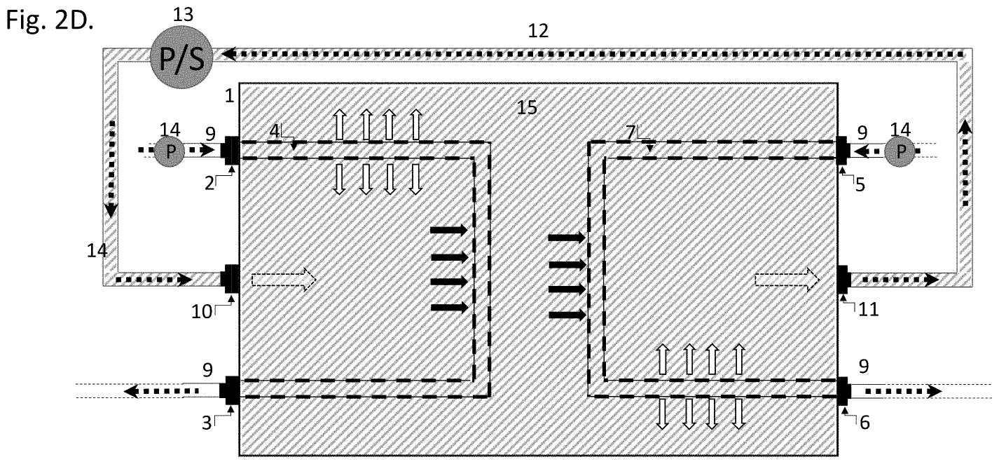

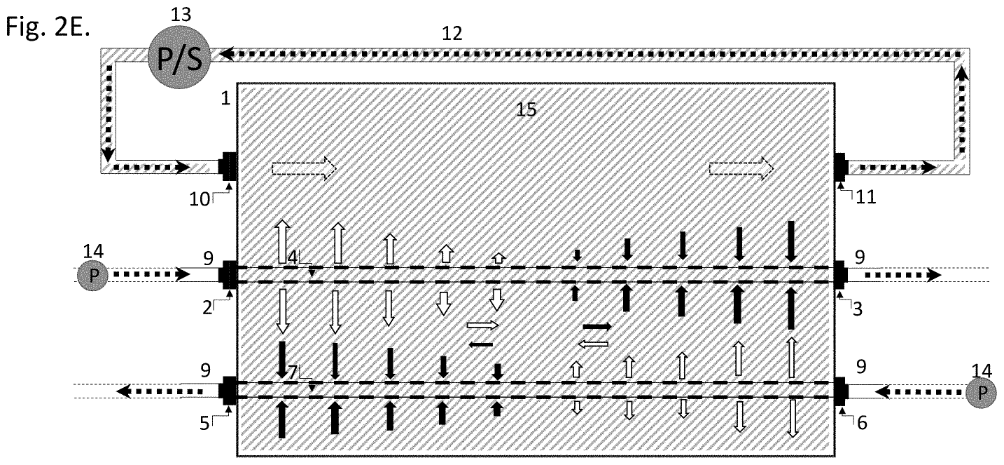

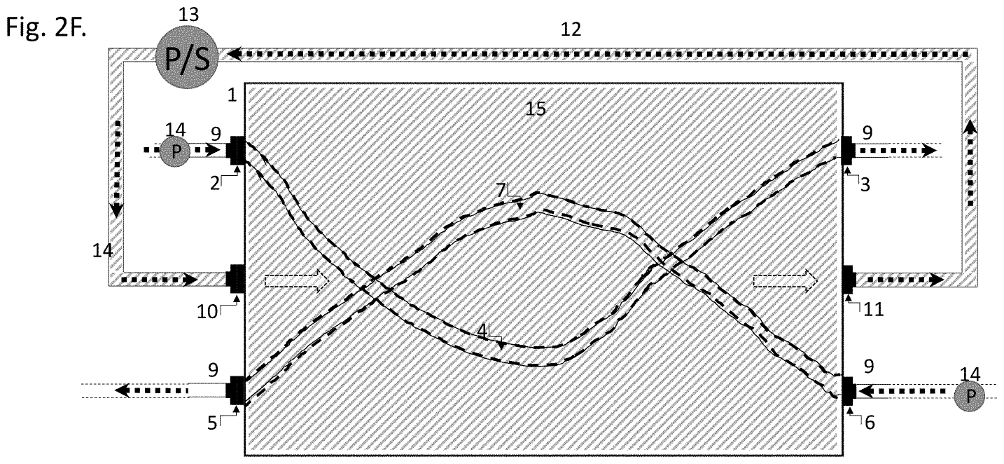

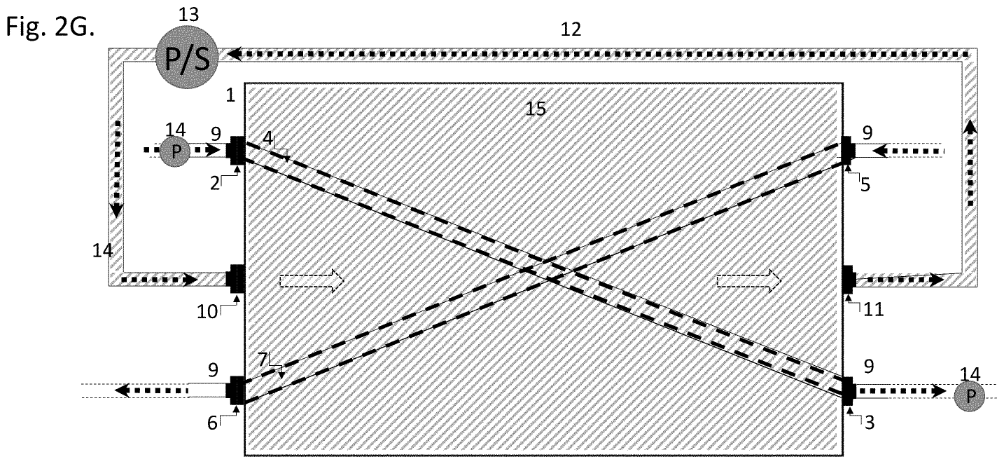

In a preferred embodiment, “compartment A” corresponds to the compartment of FIGS. 1 and 2 labelled “4”, “compartment B” as used herein corresponds to the compartment of FIGS. 1 and 2 labelled “7” and “compartment C” corresponds to the compartment of FIGS. 1 and 2 labelled “15”.

The term “membrane” as used herein refers to a physical barrier that prevents larger structures such as cells from moving from one compartment to another compartment of the perfusion chamber. Typically, the membrane is a semipermeable membrane that will allow small molecules such as salts, sugars, polysaccharides, proteins and carrier proteins, mediators, growth factors, hormones, regenerative factors, to pass through it. In other words, the semipermeable nature of the membrane ensures for these smaller molecules that a compartment is in fluid communication, i.e. in fluid flow, with the neighboring compartment even if the same fluid communication is denied or blocked for larger structures contained in the fluid that fills the compartment. A “direct fluid communication”, or “direct fluid flow”, as used herein, is preferably free of an intervening membrane. The term “fluid” refers to liquid substance and includes, in particular, physiological buffers, blood and blood plasma. The term “physiological buffer” includes for example Ringer's solution, lactated Ringer's solution, normal saline and modifications thereof including sugars and amino acids but also albumin or other larger proteins.

The term “at least one compartment A” or “at least one compartment of type A” means at least 1 compartment A but also includes at least 2, at least 3, at least 4, at least 5, at least 10, at least 15 or at least 50 compartments A. Likewise, the term “at least one compartment B” or “at least one compartment of type B” means at least 1 compartment B but also includes at least 2, at least 3, at least 4, at least 5, at least 10, at least 15 or at least 50 compartments B. In other words, the term “at least one” includes a multiplicity of said compartment A or said compartment B. Thus, the term a “multiplicity of said compartment” refers to least 2, at least 3, at least 4, at least 5, at least 10, at least 15 or at least 50 compartments. In some cases, it will be desirable to limit the number of compartments to up to 10, 20, 30, 40 or 50 compartments of type A and/or of type B.

The term “housing” or “casing” as used herein refers to a construction comprising a flexible or solid wall which provides an external structure to the perfusion chamber. The wall can comprise one or more layers, wherein these layers can be made from the same or from different materials. Examples comprise metal, glass, ceramic and organic polymers and combinations thereof. Preferred materials are selected from the group consisting of polyvinylchloride (pvc), polypropylene (PE), polyurethane (PU), polyamide, polyethylene (PE), polyethersulphone, polystyrole, silicone or silicone rubber.

According to the present teaching, compartments A and B of the blood perfusion chamber each comprise at least two “openings”, wherein one opening is specified as “first opening” and another opening is specified as “second opening”. One of these openings is typically used as an inlet into the compartment and the other opening is typically used as an outlet of the compartment. In the simplest case, the opening is or comprises a defined physical hole that provides access into the compartment. However, the hole can be combined e.g. with a tubular structure.

According to the present teaching, a “first opening” of compartment A is in direct fluid communication to a “first port” (2) of the perfusion chamber and a “second opening” of compartment A is in direct fluid communication to a “second port” (3) of the perfusion chamber. Likewise, a “first opening” of compartment B is in direct fluid communication to a “third port” (5) of the perfusion chamber and a “second opening” of compartment B is in direct fluid communication to a “fourth port” (6) of the perfusion chamber.

The present teaching encompasses embodiments defining a blood perfusion device comprising multiple “compartments A” and multiple “compartments B”. Accordingly, in these embodiments a multiplicity of “first openings” of compartment A is in direct fluid communication to a single “first port” (2) of the perfusion chamber and a multiplicity of “second openings” of compartment A is in direct fluid communication to a single “second port” (3) of the perfusion chamber. Likewise, in this embodiment, a multiplicity of “first openings” of compartment B is in direct fluid communication to a single “third port” (5) of the perfusion chamber and a multiplicity of “second openings” of compartment B is in direct fluid communication to a “fourth port” (6) of the perfusion chamber. In other words, each of said ports can be in direct fluid communication to one or more of said openings, wherein the term “one or more” preferable refers to at least 1, at least 2, at least 3, at least 4, at least 5, at least 10, at least 15 or at least 50 openings. In some cases, however, a limitation to up to 10, 20, 30, 40 or 50 openings may be desirable.

In one embodiment of the present teaching, the fluid communication between the opening of the compartment and the port of the perfusion chamber is established by, or comprises, a tube connecting the opening of the compartment to the port. In another embodiment, the compartment has, or comprises, a tapered design wherein the opening of the compartment is localized at the tip of the tapered shape. In yet another embodiment, the compartment has, or comprises, a tubular design wherein the opening of the compartment is localized at the tip of the tube.

The term “port” as used herein, essentially represents a door that allows access into the perfusion chamber. Accordingly, the port is typically integrated into the wall of the housing that provides the perfusion chamber's structure. According to the present teaching, a single port is used by one or more openings of a compartment, wherein preferably a port is used only by the same type of opening. For example, the “first port” (2) is preferably physically attached to the “first opening” of compartment A (4) and the “second port” (3) is preferably physically attached to the “second opening” of compartment A (4). In those embodiment, which comprise multiple compartments A and multiple compartments B, multiple “first openings” of compartments A are preferably attached to the “first port” (2) and multiple “second openings” of compartments B are preferably attached to the “second port” (3). Regarding compartment B (7), the “third port” (5) is preferably physically attached to the “first opening” of compartment B (7) and the “fourth port” (6) is preferably physically attached to the “second opening” of compartment B (7). In those embodiment, which comprise multiple compartments A and multiple compartments B, multiple “first openings” of compartments A are preferably attached to the “third port” (5) and multiple “second openings” of compartments B are preferably attached to the “fourth port” (6).

As used herein, the term “attached to” or “physically attached to” refers to a physical connection that allows a direct fluid communication between the opening and the port.

The ports of the blood perfusion chamber of the present teaching are typically connected outside of the perfusion chamber to tubes (9) and/or to a pump (14). The connection can comprise a flange, a nipple coupling, or a screw coupling and the like.

The term “tube” according to the present teaching includes flexible tubes and rigid tubes of any kind of suitable material, preferably medical grade material. Preferably, the tube is made from or comprises polyethylene (PE), cross-linked polyethylene (PEX), polypropylene (PP), polyvinyl chloride (PVC), nylon, natural or synthetic silicone cautchouk latex, Silastic®, Versilon®, Tygon®, Flexelene®, ethyl vinyl acetate. The tube can be made from, or comprise a thermoplastic elastomer, including for example Styrenic block copolymers (TPS, TPE-s), Thermoplastic polyolefinelastomers (TPO, TPE-o), Thermoplastic Vulcanizates (TPV, TPE-v or TPV), Thermoplastic polyurethanes (TPU, TPU), Thermoplastic copolyester (TPC, TPE-E), Thermoplastic polyamides (TPA, TPE-A), or unclassified thermoplastic elastomers (TPZ).

The term “pump” refers to any kind of pump that is suitable for extracorporeal blood perfusion. Included are, for example, centrifugal pump, fingerprint tubing pump, roller tubing pump and the like. According to the present teaching, peristaltic pumps or roller pumps are particularly preferred pumps. The pump can be a single channel pump, a dual channel pump or a multichannel pump.

The term “membrane” as used herein refers to a barrier that is semipermeable. According to the present teaching, the membrane is permeable for subcellular structures such as electrolytes, amino acids, nucleotides, lipids polysaccharides, and the like but impermeable for cells such as macrophages, dendrocytes, lymphocytes, erythrocytes and the like, including platelets. According to the present teaching, the membrane has a mean pore diameter (MPD) of 0.1-10 μm, preferably of less than 6 μm, preferably of less than 3 μm, less than 2 μm, less than 1 μm or less than 0.3 μm.

In another preferred embodiment, the membrane is characterized by a molecular weight-cutoff (MWCO) of 10 kDa-800 kDa, preferably less than 700 kDa, less than 600 kDa, less than 500 kDa, less than 400 kDa, less than 300 kDa, less than 200 kDa, less than 100 kDa, less than 500 kDa, preferably 400 kDa. However, in some embodiments it is useful to use membrane with a MWCO of at least 600 kDa, at least 500 kDa, at least 400 kDa, at least 300 kDa, at least 200 kDa, at least 100 kDa, or at least 40 kDa. The term “MWCO” refers to the molecular weight that is retained by the membrane, wherein the level of retention is at least 90%.

The membrane may be a planar membrane or a tubular hollow fiber capillary membrane. The term “planar membrane” refers to a so-called “flat sheet membrane”.

According to the present teaching, the terms “tubular membrane” and “hollow fiber membrane” are used interchangeably. The inner diameter of the tubular membrane can be constant or variable. Thus, effectively, the hollow fiber membrane system does not need to have an exact tubular shape, even though a tubular shape is preferred. The membrane is preferably microporous and can be made of any kind of suitable material. Preferred materials are selected from the group consisting of polysulfone, polyethersulfone, polypropylene, polyamide, and cellulose.

According to the present teaching, compartment A of the perfusion chamber is always separated from compartment B of the perfusion chamber by at least one membrane. The membrane is preferably configured to prevent cells which have entered compartment A from entering into compartment B. Thus, the membrane represents a physical barrier that cannot be crossed by cells.

In one embodiment, the membrane is a hollow fiber membrane system, wherein compartment A is inside a first hollow fiber membrane system that connects the first port (2) and the second port (3) and compartment B is inside a second hollow fiber membrane system that connects the third port (5) and the fourth port (6), wherein said compartment A and compartment B are thereby within compartment C (15). The term “hollow fiber membrane system” refers to a tubular structure wherein the wall of the tubular structure is formed by the membrane referred to herein above. The hollow fiber membrane system may have an inner diameter of 50-2000 μm. The term 50-2000 μm includes diameters of up to 2000 μm, up to 1000 μm, up to 500 μm. In some cases, however, a lower limit of at least 100 μm, at least 200 μm, at least 300 μm, at least 400 μm or at least 500 μm are desirable.

The lumen of the first hollow fiber membrane system represents compartment A (4) and the lumen of the second hollow fiber membrane system represents compartment B (7). This arrangement implies that compartment A is separated from compartment B by at least two membranes, wherein the first membrane is the membrane of the first hollow fiber membrane system and the second membrane is the membrane of the second hollow fiber membrane system. However, it is conceivable to use additional membranes.

The perfusion chamber of the present teaching may comprise an additional fifth port (10) and an additional sixth port (11), wherein the fifth port is in direct fluid communication to a first opening of compartment C and the sixth port is in direct fluid communication to a second opening of compartment C, wherein said ports are connected outside the perfusion chamber to a circulation system (12), preferably comprising a pump (13) for adding recirculation flow through the perfusion chamber. In one embodiment of the present teaching, the fluid communication between the opening of the compartment and the port of the perfusion chamber is established by, or comprises, a tube connecting the opening of the compartment to the port. The fluid communication between the opening of the compartment and the port may comprise a tubular structure. Preferably, the circulation system comprises at least one pump and one or more tubes.

In one embodiment, two additional planar membranes may further subdivide compartment C into compartments C1, C2 and C3, wherein the first hollow fiber membrane system may be within compartment C1 and the second hollow fiber membrane system may be within compartment C3 and wherein compartment C2 comprising the fifth port and the sixth port may be located between compartments C1 and C3.

The term “detector” as used herein refers to a device for detecting the presence or amount of a cell or of a subcellular component. The term “cell” includes any kind of blood cell such as a lymphocyte or an erythrocyte. The term “subcellular component” as used herein includes for example ions, salts, carbohydrates, saccharides, polysaccharides, carbohydrates, lipids, amino acids, nucleotides, peptides and polypeptides. Non-limiting examples of parameters that can be detected include blood cells and erythrocytes, H⁺, oxygen, carbon dioxide, C-reactive protein (CRP), hemoglobin, urea, ammonia, bilirubin. The term “detector” also includes a detector for assessing a physical state such as the temperature or pressure. The detector may comprise for an optic sensor such as a wavelength-sensitive photodiode, e.g. for the detection of red color that is presented by erythrocytes. The detection of red color that preferably refers to the detection at a wavelength of between 500 to 900 nm, more preferably at a wavelength of around 800 nm. Around 800 nm means for example at a wavelength selected from the group consisting of about 750 nm, 760 nm, 770 nm, 780 nm, 780 nm, 790 nm, 810 nm, 820 nm, 830 nm, 840 nm, and 850 nm, wherein the term “about 750 nm” and the like includes any minor modification of the wavelength by ±1-9 nm such as 749 nm, 748 nm, 747 nm, 746 nm, 745 nm, 744 nm, 743 nm, 742 nm, 741 nm, or 751 nm, 752 nm, 753 nm, 754 nm, 755 nm, 756 nm, 757 nm, 758 nm, and 759 nm.

In one embodiment, the perfusion chamber of the present teaching comprises one or more additional ports for direct injection of medical drugs, additional plasma and sensors that measure physical properties, chemical properties and substances in the device, wherein said additional ports are preferably in direct fluid communication to compartment C or to compartment C2.

The term “medical drug” includes for example heparin and antibiotics.

The term “physical property” relates to parameters described in physics, including those described in the laws of thermodynamics. E.g. a sensor that senses a color would sense a certain wavelength of light.

The term “chemical property” relates to properties of molecules, of importance, e.g., to sense glucose, lactate or pH.

The term “substance” relates to substances present in healthy or pathological plasma of individuals.

The term “control unit” relates to a component of the blood perfusion device that directs the operation of the device. Typically the control unit is associated to, comprises or controls a central processing unit and/or a computer or the like. Thus, the control unit can regulate the direction and/or rate of flow through the perfusion chamber. The control unit thus directs the activity of the pumps of the perfusion device. E.g., the control unit can measure pressure in blood lines and control a pre-set pump flow, or regulate a pre-set pump flow according to measured pressures; the control system can warm the tubing to body temperature; it can contain a heparin drug pump for control of anticoagulation; it can provide oxygen to the circuits or remove carbon dioxide or add nitric-oxide via further oxygenators in the circuit; it can feature a battery-backup.

In one embodiment blood perfusion device of the present teaching is typically operated at a flow rate of 1-250 ml/min. The flow rate is preferably controlled by the control unit. The flow rate refers to the flow through compartment A, compartment B and/or compartment C or to the flow at the first port (2), the second port (3), the third port (5), and/or the fourth port (6) of the blood perfusion chamber. A flow rate of 1-250 ml/min preferably means a flow rate of at least 1 ml/min, at least 2 ml/min, at least 3 ml/min, at least 4 ml/min, at least 5 ml/min, at least 10 ml/min, at least 15 ml/min, at least 20 ml/min, at least 40 ml/min, at least 60 ml/min, at least 80 ml/min, at least 100 ml/min, at least 120 ml/min, at least 140 ml/min, at least 160 ml/min, at least 180 ml/min or at least 200. In some cases, however, a limitation of up to 100 ml/min, up to 120 ml/min, up to 140 ml/min, up to 160 ml/min, up to 180 ml/min, up to 200 ml/min, up to 220 ml/min, up to 240 ml/min, up to 250 ml/min may be desirable. Preferred flow rates include 1-50 ml/min, 10-150 ml/min, 20-250 ml/min and 100-150 ml/min.

In one embodiment the control unit of the blood perfusion device is configured to provide a difference between the flow rate through compartment A and compartment B of less than 1% to avoid net plasma volume transfer from compartment A to the compartment B. The term “less than 1%” means for example 0.9% or less, 0.8% or less, 0.7% or less, 0.6% or less, 0.5% or less, 0.4% or less, 0.3% or less, 0.2% or less, or 0.1% or less, or no detectable difference.

In one embodiment, the control unit of the blood perfusion device is configured to provide a difference between the flow rate through compartment A and compartment B which is between 1%-20% for generating net plasma volume transfer from compartment A to compartment B. In other words, the flow rate through compartment A differs from the flow rate through compartment B by more 1% or more and 20% or less. For example, the flow rate through compartment A is up to 20% higher than the flow rate through compartment B resulting in a net plasma volume transfer from compartment A to compartment B. The terms “20% or less” and “up to 20%” mean for example up to 19%, up to 18%, up to 17%, up to 16%, up to 15%, up to 14%, up to 13%, up to 12%, up to 11%, up to 10%, up to 9%, up to 8%, up to 7%, or up to 6%.

In one embodiment, the port of the blood perfusion device of the present teaching preferably comprises or consists of pvc, polypropylene, polyamide, polyethylene, polyethersulphone, glass, or of a combination thereof. However, the port can comprise or consist of ceramic, glass or steel. The ports can represent Luer-lock and/or Dialysisis-lock configurations, the tubing connecting to the ports can have tube-clamps close to the ports in order to mechanically close a tube. Typically, the port of the blood perfusion device of the present teaching is connected to a tube. The connection may comprise for example a Dialysis-lock port.

In another embodiment, the pump of the blood perfusion device of the present teaching is selected from the group consisting of centrifugal pump (known from Plasma-separator machines), fingerprint tubing pump (known from heparin pumps, and roller tubing pump (known from dialysis machines). The pump may be any kind of peristaltic pump or positive displacement pump, preferable a roller tubing pump.

The term “adjusted by a pump” means that a pump and, optionally, the control unit described herein is used for controlling and/or maintaining the flow rate.

The present teaching also relates to method of treatment or prevention of a condition, said method comprising (a) connecting a first subject to the first port (2) and the second port (3) of the blood perfusion device of the present invention and connecting a second subject to the third port (5) and the fourth port (6) of said blood perfusion device; and (b) allowing the blood of the first subject to enter into compartment A (4) of the perfusion chamber and allowing the blood of the second subject to enter into compartment B (7) of the perfusion chamber, wherein said treatment comprises mass exchange between blood plasma of the first subject and the second subject, wherein the first subject is a healthy subject and the second subject is in need of treatment.

As used herein, the term “condition” generally refers to a state of a subject which is considered as undesirable. Also included is any state of a subject which is not considered as healthy. The condition can include one or more undesirable symptoms such as underproduction of bile for liver disease or urine for kidney disease. The condition can be characterized by one or more undesirable blood parameters such as an increased level of ammonia and bilirubin for liver disease, or urea for kidney disease or carbon dioxide for lung disease.

The term “condition”, includes age-related conditions such as low skin tone, thinning hair, brittle finger nails, muscle weakness and bone weakness. Also included are pathological conditions and diseases selected from the group consisting of hepatic failure, hepatic coma, kidney failure, kidney coma, hypercarbondioxideemia, coma, shock, sepsis.

The term “connecting a . . . subject to the . . . port” means establishing a fluid communication between a blood vessel of the subject and the port, wherein the blood vessel is typically a vein of the subject, including a peripheral vein such as the arm veins or inguinal veins, or central veins such as vena cava. An outflowing connection towards the device can also be established by arterial blood vessel access. The fluid communication can be established by using an appropriate cannula or peripheral venous catheter for entering the vein or artery. The cannula or peripheral or central catheter is connected to the port of the blood perfusion device through a tube that is connected on one end to the port and on the other end to the cannula or the venous catheter. It is preferable to use a veno-venous catheter with a double-lumen configuration inserted into a vein.

The term “mass exchange between blood plasma of the first subject and the second subject” refers to the exchange of substances or molecules in the blood plasma.

In one embodiment, the first subject is characterized by normal organ functions and normal plasma composition and the second subject is characterized by at least one deficient organ function and a deficient blood plasma composition.

The term “normal organ function” means, e.g., a typical function of, e.g., kidney, lung liver and or other organs in a healthy human subject.

The term “normal plasma composition” means, e.g., a typical composition of plasma content in a healthy human subject.

The term “deficient organ function” means, e.g., an aberrant function of an organ, e.g. liver, kidney, lung, or a tissue such as skin, muscle, cartilage or bone.

The term “deficient plasma composition” means, e.g., an aberrant composition as a consequence of malfunctioning filtration of the (kidney), metabolism (liver) or the lack of regenerative factors (ageing).

In one embodiment, the subject in need of treatment is affected from an acute, acute-on chronic or chronic liver disease associated with one or more aberrant liver-related blood parameter, wherein the parameter is preferably selected from the group consisting of ammonia, bilirubin and pH.

The term “acute liver disease” is defined as associated with a rapid onset in the range of days.

The term “chronic liver disease” is defined as associated with a slow onset in the range of month and years.

The term “acute-on liver disease” is defined as associated with a rapid onset of acute symptoms in a condition of existing chronic liver failure.

The term “aberrant liver-related blood parameter” refers to an amount of parameter, including bilirubin and ammonia, which is increased or decreased in comparison to the amount of the same parameter in a healthy subject. An increase or decrease is preferably an increase or decrease by least 20%, at least 30%, at least 50% or at least 100%.

In another embodiment, the subject in need of treatment is affected from a kidney disease, preferably an acute or chronic kidney disease associated with one or more aberrant kidney-related blood parameter, wherein the parameter is preferably selected from the group consisting of urea, water and electrolytes such as potassium, sodium, chloride.

In one embodiment, the subject in need of treatment is in need of renal dialysis. The subject is characterized by a level of urea of more than 15-20 mg/dL and in case of chronic dialysis creatinine serum levels of more than 1-2 mg/dL.

In another embodiment, the subject in need of treatment is affected from a lung disease, preferably from an acute, acute on or chronic lung disease associated with one or more aberrant lung-related blood parameter, wherein the parameter is preferably selected from the group consisting pH (7.35 to 7.45), oxygen (arterial 75-100 mmHg) and carbon dioxide (23 to 29 mEq/L.

The term “lung disease” means a decrease in oxygenation and in carbon dioxide removal in the blood of a person.

The term “acute lung disease” means an onset within days.

The term “acute on lung disease” means an onset within days on the basis of a chronic lung disease.

The term “lung-related blood parameter” includes oxygen and carbon dioxide.

In one embodiment, the subject in need of treatment is affected from a multi-organ failure.

The term “multi-organ failure” means a failure of at least two, typically at least three organs.

In one embodiment, the blood plasma of the subject in need of treatment is characterized by an aberrant level of a hormone or mediator regulating organ function, such as growth factors, mediators, cytokines and hormones that have a deviating content in comparison to the content of persons in the age of 20-30 years.

The term “hormone or mediator regulating organ function” means the homeostasis of such substances typically measured in the age range of 20-30 years.

In another embodiment, the condition is or includes a condition selected from the group consisting of a weakness of bones with bone mass loss and bone fractures (osteoporosis), a loss of muscle strength or muscle tissue (muscular dystrophies), a loss of connective tissue strength (joint cartilage weakness), a loss of hair strength and thickness, a loss of skin strength (pliability), an ageing-related condition, an integrity weakness of tissues and organs, an under-function of tissues and organs, a mal function of tissues and organs, a non-function of tissues and organs, a deregulation of oncotic pressure, a deregulation of osmolarity, an aberrant level of pH and an aberrant level of electrolyte.

In another embodiment, the condition is or includes a condition involving an aberrant level of a mediator such as a cytokine, wherein the condition is preferably shock after a trauma, septic shock after a trauma and bacteraemia), and multi-organ failure, e.g. after trauma, with a failure of at least two organs, typically three organs including lung, liver and kidney.

In another embodiment, the subject in need of treatment is a preterm baby. The term “preterm baby” refers to a baby that is born prematurely, e.g. several days and up to weeks prior the estimated day of birth.

### EXAMPLES

The examples shown in the following are exclusively illustrative and shall describe the invention in a further way. The examples shall not be construed to limit the present invention thereto.

### Example 1: Design of a Novel Blood Perfusion Device for Blood Plasma Perfusion as Alternative to Hemodialysis Dialysis Treatment of a Subject Affected from a Chronic Kidney Failure that Resides in a Rural Area

The present invention provides a blood perfusion device comprising a perfusion chamber that supports blood plasma exchange between two subjects connected to the device. The device principle is depicted for example in FIG. 2A or FIG. 2G. Each capillary membrane compartment (4) in this figure can be part of a bundle of several hundred capillary membrane compartments (4), wherein the perfusion chamber comprising the bundle can have a common inlet and a common outlet uniting the capillaries. Likewise, capillary membrane compartment (7) in this figure can be part of a bundle of several hundred capillary membrane compartments (7), wherein the perfusion chamber comprising the bundle can have a common inlet and a common outlet uniting the capillaries. In FIGS. 1 and 2, compartment (4) corresponds to “compartment A” of the present teaching, compartment (7) corresponds to “compartment B” of the present teaching and compartment (15) corresponds to “compartment C” of the present teaching.

Two independent blood circuits are connected into the device, which acts as interface between a first subject on the device (the donor) and the second subject (the recipient), who is in need of support or treatment. A first circuit starting and ending in the veins of the donor and the second starting and ending in the veins of the recipient are not directly connected, they communicate via the membrane system of the blood perfusion device which acts like an interface. Therefore, the blood perfusion device provides an immune-barrier between both blood circuits, while both blood circuits can freely exchange the molecules in the plasma.

A patient in chronic kidney failure for whom home dialysis with peritoneal dialysis machines is not sufficient anymore is in need of at least three times a day of commuting to a centralized medical dialysis facility. To avoid the weekly commute, the patient asks a relative to instead of driving him to the nearest dialysis center donate time on the mass exchanger device described herein. Provided the patient and the volunteer are blood pathogen free tested and have otherwise healthy circulatory conditions, the volunteer/donor donates time on the machine to temporarily extracorporeally provide the function of his kidneys (that typically have an overcapacity and therefore can perform kidney function for a second individual) and the patient/recipient receives the kidney function including water removal and electrolyte balancing from the donor. Preferably the donor and the recipient receive anticoagulation treatment with heparin dosages during therapy and previously received a double-lumen catheter into the arm veins from a nurse that can remain for several weeks and be reused. For four hours (depending on the previously defined dialysis need of the MD that remotely supervises the therapy) a day both can perform the therapy with the device described herein at the home of the patient, similarly to peritoneal dialysis. The disposable machine blood tubing and a disposable version of the mass exchanger described herein are connected on the device and sterilely filled with physiological electrolyte solution. The catheters of both individuals are connected to that tubing avoiding unsterility and air bubble trapping. The device pump control is switched on and the blood of both individuals perfuses through the tubing, the mass exchanger and back individually.

The membranes in the device have a molecular weight cut-off of, for example, around MW 400000. Hence, a passing of much larger blood cells from one side of the membrane to the other is prevented, while an exchange of blood plasma between all compartments of the blood perfusion chamber is allowed. Therefore, an immune-barrier is provided between both blood circuits, while both blood circuits can freely exchange those molecules in the plasma which are smaller than blood cells and also smaller than MW 400000. An important molecule to be exchanged is, e.g., the carrier protein albumin that has a MW of 60000 and that, thus, can freely pass the membrane from both sides of the blood circuit back and forth.

### Example 2: Design of a Novel Blood Perfusion Device for Blood Plasma Perfusion as Alternative to Hemodialysis Dialysis Treatment of a Subject Affected from a Chronic Kidney Failure that Resides in a Country with Underdeveloped Dialysis Treatment Infrastructure

Similarly, as described in Example 1, the same scenario can be performed in any simple medical unit in a developing country. The reason why hemodialysis is not available is the significant need and the expenses of the dialysis fluid need for hemodialysis, with first a need for availability of clean drinking water, then highly sophisticated water purification and sterilization devices with electrolyte medication, and this in amounts of around 150 L dialysis water with a need of around 250 L drinking water per session, meaning three times a week and continuously over years. To provide such patients a simpler and more affordable technical solution as alternative to hemodialysis and without the need of dialysis fluid/highly purified water that needs to be sterilized and electrolyte medicated.

To benefit from the laws of thermodynamics in physics, the natural well-characterized entropy behaviour of mass exchange is utilized through the described device configuration. This results in utilization of the forces according to the First Law of Thermodynamics, forcing molecules in blood plasma in- and out from both sides of the membrane systems in the mass exchanger device described herein. The utilization of forces according to the Second Law of Thermodynamics, the spontaneous forward transfer of molecules that are less present on the recipient patient side, an increase of entropy, is enhanced by the configuration. A pressure drop occurs along the capillary membranes within the device and transmits depending on the pore size over the membrane wall into the plasma chamber. The drop is defined as the difference in total pressure between two points of a fluid carrying network, e.g. in the beginning and the end of the lumen of a capillary, or over the membrane wall of the capillary at different points of the length of the capillary. A pressure drop occurs when frictional forces, caused by the resistance to flow, act on a fluid as it flows through the tube. Bernoulli's Principle says that as the speed of a fluid increases, the pressure of that fluid decreases. Thus, if the capillary were to remain the same size, the velocity in the capillary would decrease, resulting in an increase in pressure. Pressure change due to a velocity change. The mass exchange in the capillary membrane device configuration is enhanced over the use of a simple flat sheet membrane, as governed by various laws of physics:

In a water flowing pipe or capillary, if the diameter of a pipe is reduced, the pressure in the pipe will increase. Bernoulli says that there should be a reduction in pressure when the area is reduced. The narrower the pipe, the higher the velocity and the greater the pressure drop. Poiseuille's law says that the velocity of the steady flow of a fluid through a narrow tube (as a blood vessel or a catheter) varies directly as the pressure and the fourth power of the radius of the tube and inversely as the length of the tube and the coefficient of viscosity. According to Poiseuille's law, the flow rate through a length of pipe varies with the fourth power of the radius of the pipe. That isn't the only variable that affects flow rate; others are the length of the pipe, the viscosity of the liquid and the pressure to which the liquid is subjected.

LaPlace's Law says: The larger the vessel radius, the larger the wall tension required to withstand a given internal fluid pressure. For a given vessel radius and internal pressure, a spherical vessel will have half the wall tension of a cylindrical vessel. The membrane in the device can be characterized by the Coefficient of Permeability. It is the measure of capacity of the membrane material with which the water can easily flow through it. It is also termed as Darcy coefficient of permeability.

In this example, a device configuration is used that includes a battery pack with an automatic battery charging and battery-back up functions. This way, the device can be used over several hours without the need of regular power supply or generators in site.

### Example 3: Treatment of a Premature Infant Affected from an Acute Liver Failure

A prematurely newborn that is preterm because the mother developed antibodies against the tissues of her child has a low survival rate. A scenario that is already described in Example 1 can be applied to the father of the child, that does not show such antibodies, using the device described herein as an artificial placenta to temporarily provide the functions that the placenta in the womb of the mother cannot perform anymore. The machine is connected and operates in the same way as described in example 1.

### Example 4: Use of the Device as Regeneration Stimulating Device

In an individual of an age of 65, showing weakened bones and finger nails with bone loss and bone fractures (osteoporosis); and also loss of muscle strength or muscle tissue (muscular dystrophies), the device is used as a human-to-human regeneration device for slowing the effects of age-related diseases, to achieve anti-ageing and work-life extension. In this application, the device connects a young donor of an age of 21 years, volunteering to spend his time on the device (for a fee that compensates his efforts) with the compromised ageing recipient.

Once linked together, organs and tissues of the young donor of his time on the device equilibrate the blood level of the ageing patient, by regulating and delivering regenerative molecules. The device enables feed-back loop regulated regenerative factor and mediator exchange and their synthesis from the young to the old. Unless simple plasma donation, this shared plasma exchange in the shared circulation mimics nature's life-support system in conjoined twins. Instead of delivering frozen plasma from plasma donations, it functions like the maternal placenta that enables a shared blood circulation between the mother and the child in the womb. Like in the natural human placenta before giving birth, blood capillaries provide cross-circulation functions, where the mothers blood enables survival of her child through plasma circulation. In the device the biological capillaries of the placenta are replaced by semipermeable artificial hollow fibre capillary membrane technology. Inside the mass exchanger capillaries of the device the young and healthy donor's blood plasma safely circulates alongside the compromised or weakened plasma from the patient in need—while the described membranes define the mass exchange on a level of MW 400,000.

Regenerative molecules from the young plasma selectively flow through the semipermeable membrane systems in cases where their counterpart is missing or present on a reduced level—where they go to work immediately, reviving the compromised plasma of the patient through such a natural biological process. Blood in the one circuit that is not containing a regenerative factor, e.g. regeneration triggering testosterone in a diseased or aged patient, will be enriched if that factor is present in the other circuit, e.g. in a young healthy time donor on the device.

According to laws of physics the concentrations of any factor will try to generate an equilibrium over the membrane systems—by travelling through the membrane from the side with a large amount of factor to the side with a low amount of factor. This transfer continues until the factor is well distributed in both circuits, until a natural equilibrium is reached. In real time, the device enables to identify, regulate, and supply those regenerative mediators and factors from the young donor's blood plasma that are needed by the compromised recipient. Natural interactive biological feedback systems in the donor determine how much to up-regulate or down-regulate this biological regenerative exchange. Importantly, the device provides to the patient the donor's regenerative mediators, hormones, growth factors, and life-giving molecules while avoiding the transfer of undesired red blood cells, platelets, immune cells, and antigens.

### Example 5: Treatment of a Subject Affected from Multi-Organ Failure after a Compression Trauma Due to an Explosion on a Battlefield

Similarly to the Examples 1 and 2, the situation after severe battlefield trauma is characterized by a lack of medical facilities that can provide hemodialysis. Dialysis, the typical therapy to avoid multi-organ failure once the kidneys are injured, in such a situation is only available after sometimes day-long or week-long transfer out of the region. As alternative the device described herein can be used between soldiers that are tested for potential infectious viruses, in a way a healthy soldier provides his time to rescue the life of a second soldier that was severely injured in the battlefield and that shows medical signs of kidney nonfunction, by loss of urine production. Then the device is connected and used in the same way as in example 1. Similarly to example 2, a device configuration is used that includes a battery pack with an automatic battery charging and battery-back up functions. This way, the device can be used over several hours without the need of regular power supply, or generators, on site.

1. A blood perfusion device comprising a perfusion chamber (1) comprising at least one compartment A and at least one compartment B,
(a) compartment A (4) comprising a first opening which is in direct fluid communication to a second opening, wherein the first opening of compartment A is in direct fluid communication to a first port (2) of the perfusion chamber and the second opening of compartment A is in direct fluid communication to a second port (3) of the perfusion chamber; and
(b) compartment B (7) comprising a first opening which is in direct fluid communication to a second opening, wherein the first opening of compartment B is in direct fluid communication to a third port (5) of the perfusion chamber and the second opening of compartment B is in direct fluid communication to a fourth port (6) of the perfusion chamber;
wherein compartment A is separated from compartment B by at least one membrane, said membrane being configured to prevent cells from crossing the membrane.

2. The blood perfusion device of claim 1, wherein said ports are connected outside of the perfusion chamber to tubes (9) and/or to a pump (14).

3. The blood perfusion device of claim 1 or 2, wherein said membrane is a hollow fiber membrane system, wherein compartment A is inside a first hollow fiber membrane system that connects the first port (2) and the second port (3) and compartment B is inside a second hollow fiber membrane system that connects the third port (5) and the fourth port (6).

4. The blood perfusion device of claim 3, wherein the perfusion chamber comprises an additional fifth port (10) and an additional sixth port (11), wherein the fifth port is in direct fluid communication to a first opening of compartment C and the sixth port is in direct fluid communication to a second opening of compartment C, wherein said ports are connected outside the perfusion chamber to a circulation system (12) comprising a pump (13) for adding recirculation flow through the perfusion chamber.

5. The blood perfusion device of claim 1 or 2, wherein compartment A and compartment B are separated by at least one planar membrane.

6. The blood perfusion device of claim 5, wherein compartment A and compartment B are separated by two planar membranes, thus forming compartment C (15) between said planar membranes.

7. The blood perfusion device of claim 6, wherein the perfusion chamber comprises an additional fifth port (10) and an additional sixth port (11), wherein the fifth port is in direct fluid communication to a first opening of compartment C and the sixth port is in direct fluid communication to a second opening of compartment C, wherein said ports are connected outside the perfusion chamber to a circulation system (12) comprising a pump (13) for adding recirculation flow through the perfusion chamber.

8. The blood perfusion device of any one of claims 1 to 7, wherein the perfusion chamber, the circulation system (12) or the pump (13) comprises one or more detectors, preferably a detector for detecting pressure, rate of flow, temperature, oxygen, pH, CRP red color, hemoglobin, or blood cells.

9. The blood perfusion device of any one of claims 1 to 8, wherein the membrane is semipermeable and preferably has a molecular weight-cutoff of 10 kDa-800 kDa, more preferably at least 400 kDa.

10. The blood perfusion device of any one of claims 1 to 9, wherein the membrane comprises a material selected from the group consisting of polysulfone, polyethersulfone, polypropylene, polyamide, and cellulose, preferably polyethersulfone.

11. The blood perfusion device of any one of claims 1 to 10, wherein the casing of the perfusion chamber comprises a material selected from the group consisting of pvc, polypropylene, polyurethane, polyamide, polyethylene, polyethersulphone, polystyrole, and silicone rubber.

12. The blood perfusion device of any one of claims 3, 4 and 8 to 11, wherein the first hollow fiber membrane system and the second hollow fiber membrane system, and optionally the axis connecting the fifth port (10) and the sixth port (11) are in parallel orientation.

13. The blood perfusion device of any one of claims 1 to 12, wherein the perfusion chamber comprises one or more additional ports for direct injection of medical drugs, additional plasma and sensors that measure physical properties, chemical properties and substances in the device, wherein said additional ports are preferably in direct fluid communication to compartment C.

14. The blood perfusion device of any one of claims 1 to 13, wherein the device comprises a control unit.

15. The blood perfusion device of claim 14, wherein the control unit is configured to provide a flow rate of 1-350 ml/min at the first port (2) and the second port (3), and at the third port (5) and the fourth port (6).

16. The blood perfusion device of claim 15, wherein the flow rate is adjusted by a pump (14).

17. The blood perfusion device of any one of claims 14 to 16, wherein the control unit is configured to provide a difference between the flow rate through compartment A and compartment B of less than 1% to avoid net plasma volume transfer from compartment A to the compartment B.

18. The blood perfusion device of any one of claims 14 to 16, wherein control unit is configured to provide a difference between the flow rate through compartment A and compartment B which is between 1%-20% for generating net plasma volume transfer from compartment A to compartment B.

19. The blood perfusion device of any one of claims 1 to 18, wherein the port preferably comprises pvc, polypropylene, polyamide, polyethylene, polyethersulphone.

20. The blood perfusion device of any one of claims 1 to 19 which comprises a pump (13) or a pump (14), said pump being selected from the group consisting of centrifugal pump, fingerprint tubing pump, and roller tubing pump.

21. The blood perfusion device of any one of claims 1 to 20, which is configured to allow a flow through compartment A which is counter-directional to the flow through compartment B.

22. The blood perfusion device of any one of claims 1 to 21, which is configured so that the first opening of compartment A which is in direct fluid communication to the first port (2) is an inlet of compartment A and the second opening of compartment A which is in direct fluid communication to the second port (3) is an outlet of compartment A, and the first opening of compartment B which is in direct fluid communication to the third port (5) is an inlet of compartment B and the second opening of compartment B which is in direct fluid communication to the fourth port (6) is an outlet of compartment B.

23. The blood perfusion device of any one of claims 1 to 21, which is configured so that the first opening of compartment A which is in direct fluid communication to the first port (2) is an inlet of compartment A and the second opening of compartment A which is in direct fluid communication to the second port (3) is an outlet of compartment A, and the first opening of compartment B which is in direct fluid communication to the third port (5) is an outlet of compartment B and the second opening of compartment B which is in direct fluid communication to the fourth port (6) is an inlet of compartment B.

24. The blood perfusion device of claim 22 or 23 which is configured as defined in claim 4 or claim 7, wherein the first opening of compartment C which is in direct fluid communication to the fifth port (10) is an inlet of compartment C and the second opening of compartment C which is in direct fluid communication to the sixth port (11) is an outlet of compartment C.

25. The blood perfusion device of claim 22 or 23 which is configured as defined in claim 4 or claim 7, wherein the first opening of compartment C which is in direct fluid communication to the fifth port (10) is an outlet of compartment C and the second opening of compartment C which is in direct fluid communication to the sixth port (11) is an inlet of compartment C.

26. The blood perfusion device of any one of claims 14 to 25, wherein the control unit is configured so that the flow rate at the first port (2) and the second port (3), and/or at the third port (5) and the fourth port (6) is 1-350 ml/min.

27. The blood perfusion device of claim 26, wherein the flow rate for a subject which is a baby is 1-50 ml/min, the flow rate for a subject which is a child is 10-150 ml/min, the flow rate for a subject which is an adult is 20-350 ml/min, preferably 100-150 ml/min.

28. The blood perfusion device of claim 26 or 27, wherein the flow rate is adjusted by a pump (14).

29. The blood perfusion device of any one of claims 26 to 29, wherein the blood perfusion device is as defined in claim 4 or 7 and the flow rate through the circulation system (12) is adjusted by the pump (13).

30. Method of treatment or prevention of a condition, said method comprising
(a) connecting a first subject to the first port (2) and the second port (3) of the blood perfusion device of any one of claims 1 to 29 and connecting a second subject to the third port (5) and the fourth port (6) of said blood perfusion device; and
(b) allowing the blood of the first subject to enter into compartment A (4) of the perfusion chamber and allowing the blood of the second subject to enter into compartment B (7) of the perfusion chamber,
wherein said treatment comprises mass exchange between blood plasma of the first subject and the second subject, wherein the first subject is a healthy subject and the second subject is in need of treatment.

31. The method of claim 30, wherein the second subject is in need of blood plasma treatment.

32. The method of claim 30 or 31, wherein the first subject is characterized by normal organ functions and normal plasma composition and the second subject is characterized by at least one deficient organ function and a deficient blood plasma composition.

33. The method of claim 32, wherein the deficient organ of the second subject is the kidney, the liver or the lung.

34. The method of claim 33, wherein the liver is affected from an acute, acute-on chronic or chronic liver disease associated with one or more aberrant liver-related blood parameter, wherein the parameter is preferably selected from the group consisting of ammonia, bilirubin and pH.

35. The method of claim 33, wherein the kidney of the second subject is affected from an acute or chronic kidney disease associated with one or more aberrant kidney-related blood parameter, wherein the parameter is preferably selected from the group consisting of urea, creatinine and electrolytes including sodium, potassium, chloride, magnesium and calcium.

36. The method of claim 33 or 35, wherein the second subject is in need of renal dialysis.

37. The method of claim 33, wherein the lung is affected from an acute, acute on or chronic lung disease associated with one or more aberrant lung-related blood parameter, wherein the parameter is preferably selected from the group consisting pH, oxygen and carbon dioxide levels in the blood.

38. The method of any one of claims 30 to 37, wherein the second subject is affected from a multi-organ failure.

39. The method of any one of claims 30 to 38, wherein the blood plasma of the second subject is characterized by an aberrant level of hormone or mediator regulating organ function and where the device is used for slowing the effects of age-related diseases due to a lack of such factors, to achieve anti-ageing and work-life extension, whereas the device once linked together between a young and an old individual, enables the organs and tissues of the young donor of his time on the device to equilibrate the blood level of the ageing individual, by regulating and delivering regenerative molecules, and whereas the device enables feed-back loop regulated regenerative factor and mediator exchange and their synthesis from the young to the old; and whereas inside the mass exchanger capillaries of the device the young and healthy donor's blood plasma circulates alongside the compromised or weakened plasma from the older; and whereas regenerative molecules from the young plasma selectively flow through the semipermeable membrane systems in cases where their counterpart is missing or present on a reduced level; and whereas plasma in the one circuit that is not containing a regenerative factor, e.g. regeneration triggering testosterone in the old, will be enriched if that factor is present in the other circuit in the young healthy time donor on the device; and whereas the device provides to the old the young's regenerative mediators, hormones, growth factors, while avoiding the transfer of undesired red blood cells, platelets, and immune cells.

40. The method of any one of claims 30 to 39, wherein the condition is or includes a condition selected from the group consisting of a weakness of bones with bone loss and bone fractures (osteoporosis), a loss of muscle strength or muscle tissue (muscular dystrophies), a loss of connective tissue strength (joint cartilage weakness), a loss of hair strength and thickness, a loss of skin strength (pliability), an ageing-related condition, an integrity weakness of tissues and organs, an underfunction of tissues and organs, a mal function of tissues and organs, a non-function of tissues and organs, a deregulation of oncotic pressure, a deregulation of osmolarity, an aberrant level of pH and an aberrant level of electrolyte.

41. The method of any one of claims 30 to 40, wherein the condition is or includes a condition involving an aberrant level of a mediator such as a cytokine, wherein the condition is preferably shock, septic shock, and multi-organ failure.

42. The method of any one of claims 30 to 41, wherein the second subject is a preterm baby.
